(19) United States
(12) Patent Application Publication
(10) Pub. No.: US 2023/0045488 A1
(43) Pub. Date: Feb. 9, 2023

(54) INTRALUMINAL IMAGING BASED DETECTION AND VISUALIZATION OF INTRALUMINAL TREATMENT ANOMALIES

(71) Applicants: PHILIPS IMAGE GUIDED THERAPY CORPORATION, SAN DIEGO, CA (US); KONINKLIJKE PHILIPS N.V., Endhoven (NL)

(72) Inventors: Nikhil Sreedhar RAJGURU, SAN DIEGO, CA (US); Anuja NAIR, HIGHLAND HEIGHTS, OH (US); David CHALYAN, OCEANSIDE, CA (US)

(21) Appl. No.: 17/790,579

(22) PCT Filed: Dec. 29, 2020
(86) PCT No.: PCT/EP2020/087947

§ 371 Date: Jul. 1, 2022

Related U.S. Application Data
(60) Provisional application No. 62/957,391, filed on Jan. 6, 2020.

(51) Int. Cl. A61B 8/08; A61B 8/12; A61B 8/00

(52) U.S. Cl. CPC A61B 8/0891; A61B 8/12; A61B 8/5223; A61B 8/5246; A61B 8/463; A61B 8/0841

(57) ABSTRACT

Disclosed is an intravascular imaging system, including a processor circuit configured for communication with an intravascular imaging catheter that is sized and shaped for positioning within a lumen of a blood vessel. The processor circuit configured to receive a plurality of intravascular images obtained by the intravascular imaging catheter while the intravascular imaging catheter is positioned within the lumen, wherein the plurality of intravascular images corresponds to a plurality of locations along a length of the blood vessel. The processor is further configured to determine a measurement associated with the lumen for each image of the plurality of intravascular images, generate a curve representative of a change in the measurement along the length of the blood vessel, detect a condition of the blood vessel based on the curve, and display a graphical representation of the condition.

### TECHNICAL FIELD

The subject matter described herein relates to a system for medical imaging and data collection. In particular, the disclosed system provides a system for detecting treatment anomalies in a set of intraluminal medical images. This system has particular but not exclusive utility for diagnosis and treatment of vascular diseases.

### BACKGROUND

Various types of intraluminal (also referred to as intravascular) imaging and measurement systems are used in diagnosing and treating diseases. For example, intravascular ultrasound (IVUS) imaging is widely used in interventional cardiology as a diagnostic tool for visualizing vessels within a body of a patient. This may aid in assessing diseased vessels, such as arteries and veins within the human body, to determine the need for treatment, to optimize treatment, and/or to assess the effectiveness of treatments such as angioplasty and stenting, IVC-filter retrieval, EVAR and FEVAR (and similar on the abdominal trait) atherectomy and thrombectory. Different diseases or medical procedures produce physical features with different size, structure, density, water content, and accessibility for imaging sensors. For example, a deep-vein thrombosis (DVT) produces a clot of blood cells, whereas post-thrombotic syndrome (PTS) produces webbing or other residual structural effects in a vessel that have similar composition to the vessel wall itself, and may thus be difficult to distinguish from the vessel wall. A stent is a dense (e.g., metallic) object that may be placed in a vessel or lumen to hold the vessel or lumen open to a particular diameter. A compression occurs when anatomical structures outside the vessel or lumen impinge on the vessel or lumen, constricting it. A thrombus could occur via plaque rupture or other pathology, e.g., when blood accumulates within the lumen of a vessel due to a compression. Compression, plaque formation, and thrombus are all examples of stenosis, e.g., a narrowing of the vessel.

In some cases, intraluminal medical imaging is carried out with an IVUS device including one or more ultrasound transducers. The IVUS device may be passed into the vessel and guided to the area to be imaged. The transducers emit ultrasonic energy and receive ultrasound echoes reflected from the vessel. The ultrasound echoes are processed to create an image of the vessel of interest. The image of the vessel of interest may include one or more lesions or blockages in the vessel. A stent may be placed within the vessel to treat these blockages and intraluminal imaging may be carried out to view the placement of the stent within the vessel. Other types of treatment include thrombectomy, ablation, angioplasty, pharmaceuticals, etc.

Certain post-treatment conditions such as stent dog-boning, suboptimal stent coverage, and stent under-expansion, and natural conditions such as diffuse disease and anatomical tapering, are not detected or visualized easily in current intraluminal imaging systems.

The information included in this Background section of the specification, including any references cited herein and any description or discussion thereof, is included for technical reference purposes only and is not to be regarded as subject matter by which the scope of the disclosure is to be bound.

### SUMMARY

Disclosed is a system for advantageously detecting and displaying post-treatment anomalies within a body lumen. The current disclosure provides a system, apparatus, and method for detecting the change in value, slope, and/or gradient of lumen area, for example, over the length of the lumen, and using the gradient to detect the presence of post-treatment anomalies including stent dog-boning, stent under-dilation, suboptimal stent coverage of a lesion, and/or natural conditions such as diffuse disease and anatomical tapering. Visual identification of such anomalies may be difficult, subjective, and time consuming, whereas automated detection is fast, systematic, and repeatable. The system is hereinafter referred to as an intraluminal treatment anomaly detection system.

The intraluminal treatment anomaly detection system disclosed herein has particular, but not exclusive, utility for intraluminal ultrasound imaging procedures. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect of the intraluminal treatment anomaly detection system includes an intravascular imaging system, including: a processor circuit configured for communication with an intravascular imaging catheter sized and shaped for positioning within a lumen of a blood vessel, where the processor circuit configured to: receive a plurality of intravascular images obtained by the intravascular imaging catheter while the intravascular imaging catheter is positioned within the lumen, where the plurality of intravascular images corresponds to a plurality of locations along a length of the blood vessel; compute a dimension or determine a measurement associated with the lumen for each image of the plurality of intravascular images; generate a curve or other graphical representation representative of a change in the measurement along the length of the blood vessel; detect a condition of the blood vessel based on the curve or other graphical representation; and output, to a display in communication with the processor circuit, a graphical representation of the condition. Other embodiments of this aspect include corresponding computer systems, apparatus, and computer programs recorded on one or more computer storage devices, each configured to perform the actions of the methods.

Implementations may include one or more of the following features. The system where the processor circuit determining the measurement includes: averaging, for a location of the plurality of locations, a quantity of the measurement at the location and the quantity of the measurement at another location of the plurality of locations. The system where the processor circuit computing the dimension or determining a measurement includes the processor circuit computing or determining at least one of a cross-sectional area of the lumen or a diameter of the lumen. The system where the processor circuit detecting the condition includes the processor circuit detecting at least one of an anatomical tapering of the blood vessel or a presence of diffuse disease in the blood vessel. The system where the condition includes the anatomical tapering, and where the processor circuit detecting the condition includes the processor circuit detecting that a plaque burden of the blood vessel does not exceed a threshold value for a number of locations within a segment of the blood vessel. The system where the condition includes the diffuse disease, and where the processor circuit detecting the condition includes the processor circuit detecting that a plaque burden of the vessel exceeds a threshold value for a number of locations within a segment of the blood vessel. The system where one or more of the plurality of intravascular images includes a stent positioned within the lumen, and where the processor circuit detecting the condition of the blood vessel includes detecting a post-treatment condition. The system where the measurement includes a spacing between struts of the stent. The system where the processor circuit detecting the condition includes the processor circuit detecting at least one of dog-boning of the stent, under-dilation of the stent, or incomplete coverage of a lesion by the stent. The system where the condition is the dog-boning of the stent, and where the processor circuit detecting the condition includes the processor circuit determining that a rate of change of the measurement exhibits an inflection point within the stent, and that the rate of change of the measurement within the stent exceeds a threshold value proximal to or distal to the inflection point. The system where the condition is the under-dilation of the stent, and where the processor circuit detecting the condition includes processor circuit determining that a first value of the measurement exceeds a second value of the measurement at the edge of the stent by more than a threshold amount for a distance beyond the edge of the stent. The system where the condition is the incomplete coverage of the lesion by the stent, and where the processor circuit detecting the condition includes detecting that: for a first distance beyond an edge of the stent, a first value of the measurement is less than a second value of the measurement at the edge of the stent by at least a threshold amount; and a plaque burden for a second distance beyond the edge of the stent exceeds a threshold value. The system where the processor circuit is configured to receive an extravascular image of the blood vessel and to co-register the plurality of intravascular images to the plurality of locations along the length of the vessel in the extravascular image. The system where the processor circuit outputting the graphical representation of the condition includes the processor circuit outputting an indication of the condition along the length of the vessel in the extravascular image. The system further including: the intravascular imaging catheter, where the intravascular imaging catheter includes an intravascular ultrasound (IVUS) imaging catheter. Implementations of the described techniques may include hardware, a method or process, or computer software on a computer-accessible medium.

One general aspect includes an intravascular imaging method, including: receiving, at a processor circuit in communication with an intravascular imaging catheter, a plurality of intravascular images obtained by the intravascular imaging catheter while the intravascular imaging catheter is positioned with a lumen a blood vessel, where the plurality of intravascular images corresponds to a plurality of locations along a length of the blood vessel; computing a dimension or determining a measurement, by the processor circuit, associated with the lumen for each image of the plurality of intravascular images; generating, by the processor circuit, a curve or graphical representation representative of a change in the measurement along the length of the blood vessel; detecting, by the processor circuit, a condition of the blood vessel based on the curve or graphical representation; and outputting, to a display in communication with the processor circuit, a graphical representation of the condition. Other embodiments of this aspect include corresponding computer systems, apparatus, and computer programs recorded on one or more computer storage devices, each configured to perform the actions of the methods.

One general aspect includes an intravascular ultrasound (IVUS) imaging system, comprising: a processor circuit configured for communication with an IVUS imaging catheter sized and shaped for positioning within a lumen of a blood vessel, wherein the processor circuit configured to: receive a plurality of IVUS images obtained by the IVUS imaging catheter while the IVUS imaging catheter is positioned within the lumen, wherein the plurality of IVUS images corresponds to a plurality of locations along a length of the blood vessel; determine a measurement associated with the lumen for each image of the plurality of IVUS images; generate a curve representative of a change in the measurement along the length of the blood vessel; detect a condition of the blood vessel based on the curve, wherein the condition comprises at least one of dog-boning of a stent within the blood vessel, under-dilation of the stent, or incomplete coverage of a lesion of the blood vessel by the stent, diffuse disease, or anatomical tapering; and output, to a display in communication with the processor circuit, a graphical representation representative of the condition.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the intraluminal treatment anomaly detection system, as defined in the claims, is provided in the following written description of various embodiments of the disclosure and illustrated in the accompanying drawings.

### BRIEF DESCRIPTION OF THE DRAWINGS

Illustrative embodiments of the present disclosure will be described with reference to the accompanying drawings, of which:

FIG. 1 is a diagrammatic schematic view of an intraluminal imaging system, according to aspects of the present disclosure.

FIG. 2 illustrates a blood vessel incorporating a stenosis.

FIG. 3 illustrates a blood vessel incorporating a stenosis and dilated with a stent.

FIG. 4 shows an example screen display of an intraluminal imaging system in accordance with aspects of the present disclosure.

FIG. 5 shows an example screen display of an intraluminal imaging system in accordance with at least one embodiment of the present disclosure.

FIG. 6 shows an example screen display of an intraluminal imaging system in accordance with at least one embodiment of the present disclosure.

FIG. 7 shows a schematic view of a vessel whose vessel walls have been dilated with a stent having a proximal edge and a distal edge, in accordance with aspects of the present disclosure.

FIG. 8 is a flow diagram illustrating the steps executed by an example intraluminal treatment anomaly detection system in accordance with at least one embodiment of the present disclosure.

FIG. 9 is a flow diagram of an example stent under-dilation detection algorithm in accordance with at least one embodiment of the present disclosure.

FIG. 10 is a flow diagram of an example dog-boning detection algorithm in accordance with at least one embodiment of the present disclosure.

FIG. 11 is a flow diagram of a different example dog-boning detection algorithm 870 in accordance with at least one embodiment of the present disclosure.

FIG. 12 is a flow diagram of an example suboptimal stent placement detection algorithm 880 in accordance with at least one embodiment of the present disclosure.

FIG. 13 is a flow diagram of an example anatomical tapering vs. diffuse disease detection algorithm in accordance with at least one embodiment of the present disclosure.

FIG. 14 is a schematic diagram of a processor circuit, according to embodiments of the present disclosure.

FIG. 15 shows a schematic view of a vessel whose vessel walls have been dilated with a stent that exhibits dog-boning, in accordance with aspects of the present disclosure.

### DETAILED DESCRIPTION

The present disclosure relates generally to medical imaging, including imaging associated with a body lumen of a patient using an intraluminal imaging device. In some cases, intraluminal imaging is carried out with an IVUS device including one or more ultrasound transducers. The IVUS device may be passed into the vessel and guided to the area to be imaged. The transducers emit ultrasonic energy and receive ultrasound echoes reflected from the vessel. The ultrasound echoes are processed to create an image of the vessel of interest. The image of the vessel of interest may include one or more lesions or blockages in the vessel. A stent may be placed within the vessel to treat these blockages, and intraluminal imaging may be carried out to view the placement of the stent within the vessel. Other types of treatment include thrombectomy, ablation, angioplasty, pharmaceuticals, etc.

Disclosed is a system for advantageously detecting and displaying post-treatment anomalies within a body lumen such as a blood vessel. The current disclosure provides a system, apparatus, and method for detecting the gradient of lumen area over the length of the lumen, and using the gradient to detect the presence of anomalies including dog-boning, suboptimal coverage, and diffuse disease. The anomalies described in this disclosure, including stent dog-boning, suboptimal stent coverage (e.g., incomplete coverage of a lesion by the stent), diffuse disease, and anatomical tapering are not visualized easily in current intraluminal imaging devices, and visualizing these features clearly and automatically represents a substantial time savings for clinicians and may also contribute to timely and effective treatment. Accurate disease detection or anomaly detection can influence not only stenting decisions but also treatment steps such as choice of balloon, or choice of other therapeutic devices including but not limited to atherectomy devices. The logic and algorithms disclosed herein can be used in the review of any automated measurement system that is used for example in pre-PCI and post-PCI case analysis. The system can also be used in the education and training of novice users. The system is hereinafter referred to as an intraluminal treatment anomaly detection system.

The devices, systems, and methods described herein can include one or more features described in U.S. Provisional App. No. 62/750,983 (Attorney Docket No. 2018PF01112-44755.2000PV01), filed 26 Oct. 2018, U.S. Provisional App. No. 62/751,268 (Attorney Docket No. 2018PF01160-44755.1997PV01), filed 26 Oct. 2018, U.S. Provisional App. No. 62/751,289 (Attorney Docket No. 2018PF01159-44755.1998PV01), filed 26 Oct. 2018, U.S. Provisional App. No. 62/750,996 (Attorney Docket No. 2018PF01145-44755.1999PV01), filed 26 Oct. 2018, U.S. Provisional App. No. 62/751,167 (Attorney Docket No. 2018PF01115-44755.2000PV01), filed 26 Oct. 2018, and U.S. Provisional App. No. 62/751,185 (Attorney Docket No. 2018PF01116-44755.2001PV01), filed 26 Oct. 2018, each of which is hereby incorporated by reference in its entirety as though fully set forth herein.

The devices, systems, and methods described herein can also include one or more features described in U.S. Provisional App. No. 62/642,847 (Attorney Docket No. 2017PF02103), filed Mar. 14, 2018 (and a Non-Provisional Application filed therefrom on Mar. 12, 2019 as U.S. Ser. No. 16/351,175), U.S. Provisional App. No. 62/712,009 (Attorney Docket No. 2017PF02296), filed Jul. 30, 2018, U.S. Provisional App. No. 62/711,927 (Attorney Docket No. 2017PF02101), filed Jul. 30, 2018, and U.S. Provisional App. No. 62/643,366 (Attorney Docket No. 2017PF02365), filed Mar. 15, 2018 (and a Non-Provisional Application filed therefrom on Mar. 15, 2019 as U.S. Ser. No. 16/354,970), each of which is hereby incorporated by reference in its entirety as though fully set forth herein.

The present disclosure substantially aids a clinician in identifying treatment anomalies within a vessel using the data available in an intraluminal pullback image sequence, by computing and graphing a filtered gradient of at least one per-frame metric related to the images. Implemented on a medical imaging console (e.g., an IVUS imaging console) in communication with a medical imaging sensor (e.g., an intraluminal ultrasound sensor), the intraluminal treatment anomaly detection system disclosed herein provides both time savings and an improvement in the detection certainty and location certainty of particular anomaly types. This improved methodology transforms an imprecise, judgment-driven procedure into a quantitative, repeatable process that requires fewer and simpler steps to be taken by the clinician or other user. This occurs for example without the normally routine need to apply human judgment or vision to estimate where in a lumen an anomaly may be present. This unconventional approach improves the functioning of the medical imaging console and sensor, by standardizing and automating the detection criteria for the anomalies.

The intraluminal treatment anomaly detection system may be implemented as a set of logical branches and mathematical operations, whose outputs are viewable on a display, and operated by a control process executing on a processor that accepts user inputs (e.g., from a user interface such as a keyboard, mouse, or touchscreen interface), and that is in communication with one or more medical imaging sensors (e.g., intraluminal ultrasound sensors). In that regard, the control process performs certain specific operations in response to different inputs or selections made by a user at the start of an imaging procedure, and may also respond to inputs made by the user during the procedure. Certain structures, functions, and operations of the processor, display, sensors, and user input systems are known in the art, while others are recited herein to enable novel features or aspects of the present disclosure with particularity.

These descriptions are provided for exemplary purposes only, and should not be considered to limit the scope of the intraluminal treatment anomaly detection system. Certain features may be added, removed, or modified without departing from the spirit of the claimed subject matter.

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

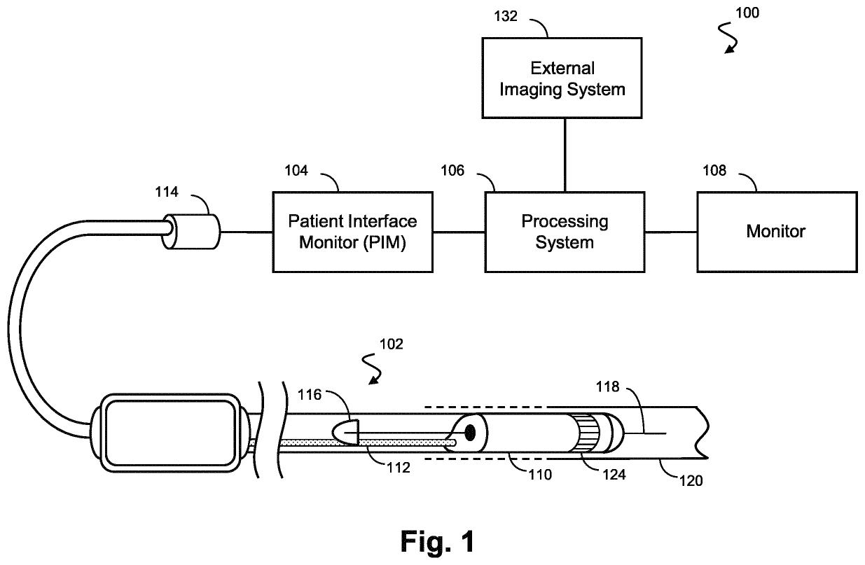

FIG. 1 is a diagrammatic schematic view of an intraluminal imaging system incorporating the intraluminal treatment anomaly detection system, according to aspects of the present disclosure. The intraluminal imaging system 100 can be an intravascular ultrasound (IVUS) imaging system in some embodiments. The intraluminal imaging system 100 may include an intraluminal device 102, a patient interface module (PIM) 104, a console or processing system 106, a monitor 108, and an external imaging system 132 which may include angiography, ultrasound, X-ray, computed tomography (CT), magnetic resonance imaging (MRI), or other imaging technologies, equipment, and methods. The intraluminal device 102 is sized and shaped, and/or otherwise structurally arranged to be positioned within a body lumen of a patient. For example, the intraluminal device 102 can be a catheter, guide wire, guide catheter, pressure wire, and/or flow wire in various embodiments. In some circumstances, the system 100 may include additional elements and/or may be implemented without one or more of the elements illustrated in FIG. 1. For example, the system 100 may omit the external imaging system 132.

The intraluminal imaging system 100 (or intravascular imaging system) can be any type of imaging system suitable for use in the lumens or vasculature of a patient. In some embodiments, the intraluminal imaging system 100 is an intraluminal ultrasound (IVUS) imaging system. In other embodiments, the intraluminal imaging system 100 may include systems configured for forward looking intraluminal ultrasound (FL-IVUS) imaging, intraluminal photoacoustic (IVPA) imaging, intracardiac echocardiography (ICE), transesophageal echocardiography (TEE), and/or other suitable imaging modalities.

It is understood that the system 100 and/or device 102 can be configured to obtain any suitable intraluminal imaging data. In some embodiments, the device 102 may include an imaging component of any suitable imaging modality, such as optical imaging, optical coherence tomography (OCT), etc. In some embodiments, the device 102 may include any suitable non-imaging component, including a pressure sensor, a flow sensor, a temperature sensor, an optical fiber, a reflector, a mirror, a prism, an ablation element, a radio frequency (RF) electrode, a conductor, or combinations thereof. Generally, the device 102 can include an imaging element to obtain intraluminal imaging data associated with the lumen 120. The device 102 may be sized and shaped (and/or configured) for insertion into a vessel or lumen 120 of the patient.

The system 100 may be deployed in a catheterization laboratory having a control room. The processing system 106 may be located in the control room. Optionally, the processing system 106 may be located elsewhere, such as in the catheterization laboratory itself. The catheterization laboratory may include a sterile field while its associated control room may or may not be sterile depending on the procedure to be performed and/or on the health care facility. The catheterization laboratory and control room may be used to perform any number of medical imaging procedures such as angiography, fluoroscopy, CT, IVUS, virtual histology (VH), forward looking IVUS (FL-IVUS), intraluminal photoacoustic (IVPA) imaging, a fractional flow reserve (FFR) determination, a coronary flow reserve (CFR) determination, optical coherence tomography (OCT), computed tomography, intracardiac echocardiography (ICE), forward-looking ICE (FLICE), intraluminal palpography, transesophageal ultrasound, fluoroscopy, and other medical imaging modalities, or combinations thereof. In some embodiments, device 102 may be controlled from a remote location such as the control room, such than an operator is not required to be in close proximity to the patient.

The intraluminal device 102, PIM 104, monitor 108, and external imaging system 132 may be communicatively coupled directly or indirectly to the processing system 106. These elements may be communicatively coupled to the medical processing system 106 via a wired connection such as a standard copper link or a fiber optic link and/or via wireless connections using IEEE 802.11 Wi-Fi standards, Ultra Wide-Band (UWB) standards, wireless FireWire, wireless USB, or another high-speed wireless networking standard. The processing system 106 may be communicatively coupled to one or more data networks, e.g., a TCP/IP-based local area network (LAN). In other embodiments, different protocols may be utilized such as Synchronous Optical Networking (SONET). In some cases, the processing system 106 may be communicatively coupled to a wide area network (WAN). The processing system 106 may utilize network connectivity to access various resources. For example, the processing system 106 may communicate with a Digital Imaging and Communications in Medicine (DICOM) system, a Picture Archiving and Communication System (PACS), and/or a Hospital Information System (HIS) via a network connection.

At a high level, an ultrasound imaging intraluminal device 102 emits ultrasonic energy from a transducer array 124 included in scanner assembly 110 mounted near a distal end of the intraluminal device 102. The ultrasonic energy is reflected by tissue structures in the medium (such as a lumen 120) surrounding the scanner assembly 110, and the ultrasound echo signals are received by the transducer array 124. The scanner assembly 110 generates electrical signal(s) representative of the ultrasound echoes. The scanner assembly 110 can include one or more single ultrasound transducers and/or a transducer array 124 in any suitable configuration, such as a planar array, a curved array, a circumferential array, an annular array, etc. For example, the scanner assembly 110 can be a one-dimensional array or a two-dimensional array in some instances. In some instances, the scanner assembly 110 can be a rotational ultrasound device. The active area of the scanner assembly 110 can include one or more transducer materials and/or one or more segments of ultrasound elements (e.g., one or more rows, one or more columns, and/or one or more orientations) that can be uniformly or independently controlled and activated. The active area of the scanner assembly 110 can be patterned or structured in various basic or complex geometries. The scanner assembly 110 can be disposed in a side-looking orientation (e.g., ultrasonic energy emitted perpendicular and/or orthogonal to the longitudinal axis of the intraluminal device 102) and/or a forward-looking looking orientation (e.g., ultrasonic energy emitted parallel to and/or along the longitudinal axis). In some instances, the scanner assembly 110 is structurally arranged to emit and/or receive ultrasonic energy at an oblique angle relative to the longitudinal axis, in a proximal or distal direction. In some embodiments, ultrasonic energy emission can be electronically steered by selective triggering of one or more transducer elements of the scanner assembly 110.

The ultrasound transducer(s) of the scanner assembly 110 can be a piezoelectric micromachined ultrasound transducer (PMUT), capacitive micromachined ultrasonic transducer (CMUT), single crystal, lead zirconate titanate (PZT), PZT composite, other suitable transducer type, and/or combinations thereof. In an embodiment the ultrasound transducer array 124 can include any suitable number of individual transducer elements or acoustic elements between 1 acoustic element and 1000 acoustic elements, including values such as 2 acoustic elements, 4 acoustic elements, 36 acoustic elements, 64 acoustic elements, 128 acoustic elements, 500 acoustic elements, 812 acoustic elements, and/or other values both larger and smaller.

The PIM 104 transfers the received echo signals to the processing system 106 where the ultrasound image (including the flow information) is reconstructed and displayed on the monitor 108. The console or processing system 106 can include a processor and a memory. The processing system 106 may be operable to facilitate the features of the intraluminal imaging system 100 described herein. For example, the processor can execute computer readable instructions stored on the non-transitory tangible computer readable medium.

The PIM 104 facilitates communication of signals between the processing system 106 and the scanner assembly 110 included in the intraluminal device 102. This communication may include providing commands to integrated circuit controller chip(s) within the intraluminal device 102, selecting particular element(s) on the transducer array 124 to be used for transmit and receive, providing the transmit trigger signals to the integrated circuit controller chip(s) to activate the transmitter circuitry to generate an electrical pulse to excite the selected transducer array element(s), and/or accepting amplified echo signals received from the selected transducer array element(s) via amplifiers included on the integrated circuit controller chip(s). In some embodiments, the PIM 104 performs preliminary processing of the echo data prior to relaying the data to the processing system 106. In examples of such embodiments, the PIM 104 performs amplification, filtering, and/or aggregating of the data. In an embodiment, the PIM 104 also supplies high- and low-voltage DC power to support operation of the intraluminal device 102 including circuitry within the scanner assembly 110.

The processing system 106 receives echo data from the scanner assembly 110 by way of the PIM 104 and processes the data to reconstruct an image of the tissue structures in the medium surrounding the scanner assembly 110. Generally, the device 102 can be utilized within any suitable anatomy and/or body lumen of the patient. The processing system 106 outputs image data such that an image of the vessel or lumen 120, such as a cross-sectional IVUS image of the lumen 120, is displayed on the monitor 108. Lumen 120 may represent fluid filled or fluid-surrounded structures, both natural and man-made. Lumen 120 may be within a body of a patient. Lumen 120 may be a blood vessel, such as an artery or a vein of a patient's vascular system, including cardiac vasculature, peripheral vasculature, neural vasculature, renal vasculature, and/or or any other suitable lumen inside the body. For example, the device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the device 102 may be used to examine man-made structures such as, but without limitation, heart valves, stents, shunts, filters and other devices.

The controller or processing system 106 may include a processing circuit having one or more processors in communication with memory and/or other suitable tangible computer readable storage media. The controller or processing system 106 may be configured to carry out one or more aspects of the present disclosure. In some embodiments, the processing system 106 and the monitor 108 are separate components. In other embodiments, the processing system 106 and the monitor 108 are integrated in a single component. For example, the system 100 can include a touch screen device, including a housing having a touch screen display and a processor. The system 100 can include any suitable input device, such as a touch sensitive pad or touch screen display, keyboard/mouse, joystick, button, etc., for a user to select options shown on the monitor 108. The processing system 106, the monitor 108, the input device, and/or combinations thereof can be referenced as a controller of the system 100. The controller can be in communication with the device 102, the PIM 104, the processing system 106, the monitor 108, the input device, and/or other components of the system 100.

In some embodiments, the intraluminal device 102 includes some features similar to traditional solid-state IVUS catheters, such as the EagleEye® catheter available from Volcano Corporation and those disclosed in U.S. Pat. No. 7,846,101 hereby incorporated by reference in its entirety. For example, the intraluminal device 102 may include the scanner assembly 110 near a distal end of the intraluminal device 102 and a transmission line bundle 112 extending along the longitudinal body of the intraluminal device 102. The cable or transmission line bundle 112 can include a plurality of conductors, including one, two, three, four, five, six, seven, or more conductors.

The transmission line bundle 112 terminates in a PIM connector 114 at a proximal end of the intraluminal device 102. The PIM connector 114 electrically couples the transmission line bundle 112 to the PIM 104 and physically couples the intraluminal device 102 to the PIM 104. In an embodiment, the intraluminal device 102 further includes a guidewire exit port 116. Accordingly, in some instances the intraluminal device 102 is a rapid-exchange catheter. The guidewire exit port 116 allows a guidewire 118 to be inserted towards the distal end in order to direct the intraluminal device 102 through the lumen 120.

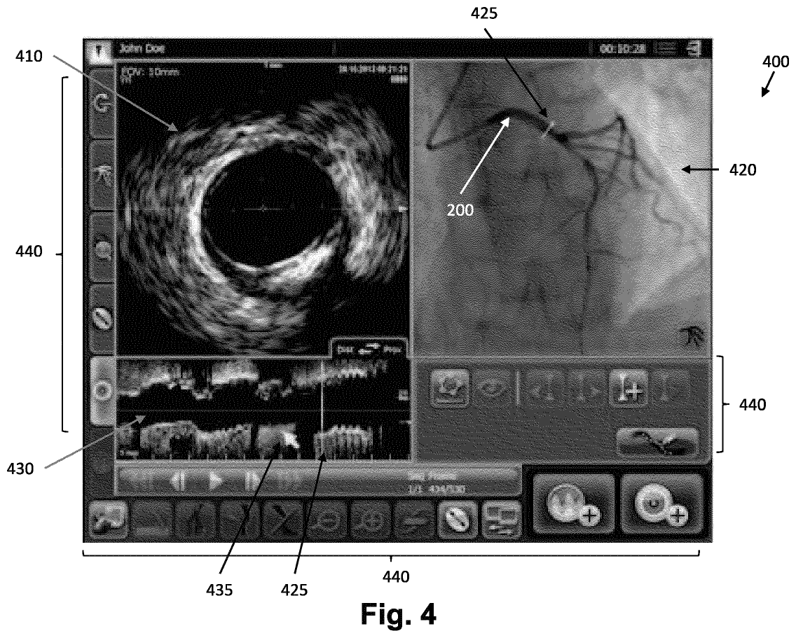

The monitor 108 may be a display device such as a computer monitor or other type of screen. The monitor 108 may be used to display selectable prompts, instructions, and visualizations of imaging data to a user. In some embodiments, the monitor 108 may be used to provide a procedure-specific workflow to a user to complete an intraluminal imaging procedure. This workflow may include performing a pre-stent plan to determine the state of a lumen and potential for a stent, as well as a post-stent inspection to determine the status of a stent that has been positioned in a lumen. The workflow may be presented to a user as any of a variety of different the displays or visualizations (as shown for example in FIGS. 4-6).

The external imaging system 132 can be configured to obtain x-ray, radiographic, angiographic (e.g., with contrast), and/or fluoroscopic (e.g., without contrast) images of the body of a patient (including the vessel 120). External imaging system 132 may also be configured to obtain computed tomography images of the body of patient (including the vessel 120). The external imaging system 132 may include an external ultrasound probe configured to obtain ultrasound images of the body of the patient (including the vessel 120) while positioned outside the body. In some embodiments, the system 100 includes other imaging modality systems (e.g., MRI) to obtain images of the body of the patient (including the vessel 120). The processing system 106 can utilize the images of the body of the patient in conjunction with the intraluminal images obtained by the intraluminal device 102.

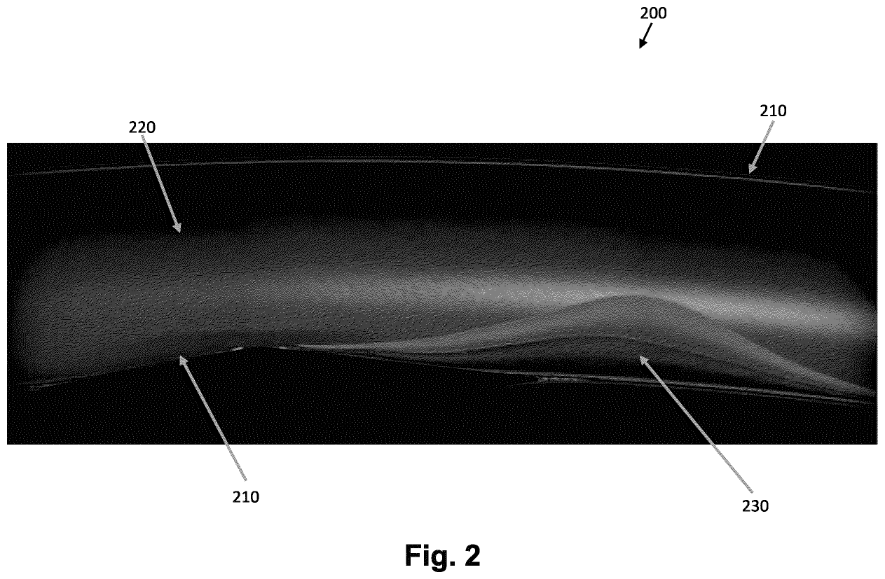

FIG. 2 illustrates a blood vessel 200 incorporating a stenosis 230. The stenosis 230 may occur inside the vessel walls (e.g., a thrombus, clot, or plaque) or outside the vessel walls 210 (e.g., a compression), and may restrict the flow of blood 220. Compression may be caused by other anatomical structures outside the blood vessel 200, including but not limited to a tendon, ligament, or neighboring lumen.

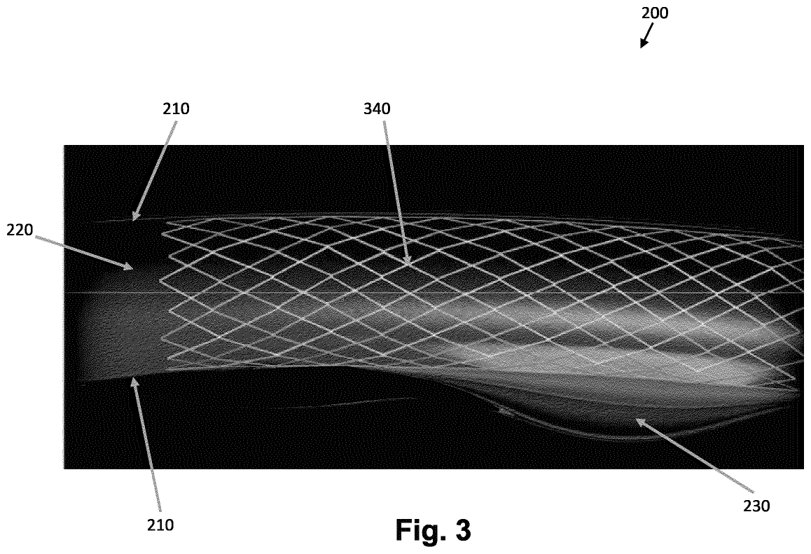

FIG. 3 illustrates a blood vessel 200 incorporating a stenosis 230 and dilated with a stent 340. The stent 340 displaces and arrests the stenosis 230, pushing the vessel walls 310 outward, thus reducing the flow restriction for the blood 220. Other treatment options for alleviating an occlusion may include but are not limited to thrombectomy, ablation, angioplasty, and pharmaceuticals. However, in a large majority of cases it may be highly desirable to obtain accurate and timely intravascular images of the affected area, along with accurate and detailed knowledge of the location, orientation, length, and volume of the affected area prior to, during, or after treatment.

FIG. 4 shows an example screen display 400 of an intraluminal imaging system 100 in accordance with aspects of the present disclosure. The screen display 400 includes a tomographic intravascular image 410 (e.g., an IVUS image) of a vessel 200, an externally acquired roadmap image 420 (e.g., an x-ray fluoroscopic image) of the same vessel 200, and an image longitudinal display (ILD) 430, which comprises longitudinal cross sections of a plurality of tomographic intravascular images 410. In this example, the roadmap image 420 and ILD 430 each include a position marker 425 showing the current (co-registered) position of the intraluminal probe 102 (and hence of the tomographic image 410) within the vessel 200. The ILD 430 also includes a region-of-interest marker 435 that may, for example, identify the location of a diseased section of the vessel 200. Co-registration of the intraluminal images 410 with the roadmap image 420 permits a clinician or other user to see, at a glance, precisely where in the vessel 200 the intraluminal imaging probe 102 is currently imaging. This position certainty may be associated with improved clinical outcomes. Aspects of co-registration are described, for example, in U.S. Pat. Nos. 7,930,014 and 8,298,147, the entireties of which are hereby incorporated by reference in its eternity.

Also visible in this example are a plurality of user interface controls 440.

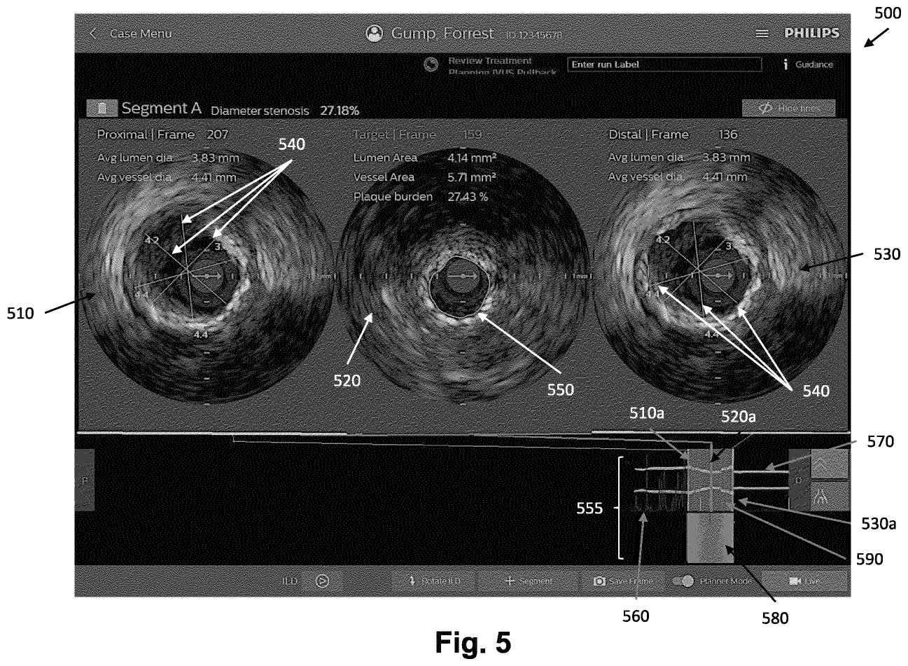

FIG. 5 shows an example screen display 500 of an intraluminal imaging system 100 in accordance with at least one embodiment of the present disclosure. The screen display 500 includes three cross-sectional images (e.g., axial or radial cross-sectional images, also known as tomographic images): a proximal reference frame 510, a target frame 520, and a distal reference frame 530. In an example, the proximal reference frame 510 and distal reference frame 530 represent healthy tissue proximal and distal to a stenosis or other constriction in the lumen 120 (e.g., a blood vessel 200), and the healthy status of the tissue is indicated by diameter measurements 540, which can be used to determine the cross-sectional area of the lumen 120. In an example, the target frame 520 represents the narrowest portion of a diseased segment of the lumen 120, as shown by the detected lumen border or perimeter 550, which can be used to determine the diameter or cross-sectional area of the lumen 120.

Examples of border detection, image processing, image analysis, and/or pattern recognition include U.S. Pat. No. 6,200,268 entitled “VASCULAR PLAQUE CHARACTERIZATION” issued Mar. 13, 2001 with D. Geoffrey Vince, Barry D. Kuban and Anuja Nair as inventors, U.S. Pat. No. 6,381,350 entitled “INTRAVASCULAR ULTRASONIC ANALYSIS USING ACTIVE CONTOUR METHOD AND SYSTEM” issued Apr. 30, 2002 with Jon D. Klingensmith, D. Geoffrey Vince and Raj Shekhar as inventors, U.S. Pat. No. 7,074,188 entitled “SYSTEM AND METHOD OF CHARACTERIZING VASCULAR TISSUE” issued Jul. 11, 2006 with Anuja Nair, D. Geoffrey Vince, Jon D. Klingensmith and Barry D. Kuban as inventors, U.S. Pat. No. 7,175,597 entitled “NON-INVASIVE TISSUE CHARACTERIZATION SYSTEM AND METHOD” issued Feb. 13, 2007 with D. Geoffrey Vince, Anuja Nair and Jon D. Klingensmith as inventors, U.S. Pat. No. 7,215,802 entitled “SYSTEM AND METHOD FOR VASCULAR BORDER DETECTION” issued May 8, 2007 with Jon D. Klingensmith, Anuja Nair, Barry D. Kuban and D. Geoffrey Vince as inventors, U.S. Pat. No. 7,359,554 entitled “SYSTEM AND METHOD FOR IDENTIFYING A VASCULAR BORDER” issued Apr. 15, 2008 with Jon D. Klingensmith, D. Geoffrey Vince, Anuja Nair and Barry D. Kuban as inventors and U.S. Pat. No. 7,463,759 entitled “SYSTEM AND METHOD FOR VASCULAR BORDER DETECTION” issued Dec. 9, 2008 with Jon D. Klingensmith, Anuja Nair, Barry D. Kuban and D. Geoffrey Vince, as inventors, the teachings of which are hereby incorporated by reference herein in their entirety.

Also visible is a longitudinal display 555 that includes a probability-of-stent graph 560 with probability on the Y axis and longitudinal position on the X-axis. A low or zero probability-of-stent value for a given position indicates that no highly dense (e.g., metallic) objects were detected in the tomographic frame captured at that location, whereas a high value indicates multiple detections of high-density points that may represent the struts of a metallic stent 340. Pattern recognition, either of this graph or of the tomographic images themselves, can be used to detect the proximal and distal edges of a stent 340 that has been placed within the lumen 120. In the example shown in the figure, position markers 510a, 520a, and 530a mark the locations on the longitudinal display 555 where of the proximal reference frame 510, target frame 520, and distal reference frame 530 were captured within the vessel, along with a graphical diameter or area indicator 570 that is symmetric around an invisible horizontal centerline. The diameter or area indicator 570 shows the lumen diameter at each point, as determined from the tomographic images 410 taken at each location within the lumen 120. In an example, the diameter or area indicator 570 is smoothed (e.g., shows an average of the current frame and the surrounding 2, 4, or 6 frames) in order to reduce the effect of normal frame-to-frame variability in the diameter or area measurement. In some instances, the longitudinal display could be a stack of tomographic image frames so that the actual lumen contour is shown. In this case, the lumen contour need not be symmetric, as it follows the actual contours of the vessel, as shown for example in the ILD 430 of FIG. 4. In other instances, the longitudinal display could be a graphical representation of the actual lumen contour, which again may not necessarily be symmetric.

Also visible beneath the diameter indicator 570 of the longitudinal display 555 is a longitudinal gradient display 580, which indicates the slope or gradient of the diameter indicator at points along the longitudinal display 555. In an example, values of the diameter indicator 570 that exceed a positive threshold value are indicated on the gradient indicator 580 with varying shades, brightness, or intensity of a first color, whereas slope or gradient values that exceed a negative threshold are indicated with varying shades, brightness, or intensity of a second color.

Dog-boning is a condition that occurs when a stent 340 has been dilated to a greater diameter at the ends than at the center, and depending on severity this may be considered an anomalous or sub-optimal treatment result that requires correction. In the example shown in FIG. 5, the probability-of-stent graph indicates the presence of a stent 340, and both the diameter or area indicator 570 and the gradient indicator 580 of the longitudinal display 555 indicate the dog-boning within the stent 340, as the diameter or area 570 is visibly narrower at the target frame 520 than at the proximal reference frame 510 or distal reference frame 530, and the gradient 580 is negative between the proximal reference frame 510 and the target frame 520, but positive between the target frame 520 and the distal reference frame 530. Therefore, in this example, a shaded dog-boning warning indicator 590 has been overlaid onto the longitudinal display 555.

Based for example on the dog-boning warning indicator 590, or on the colors presented by the gradient display 580, or on the diameters shown in the diameter indicator 570, a clinician may be able to see at a glance whether this dog-boning is serious enough to require correction (e.g., by reinserting a noncompliant balloon and expanding it in the vicinity of the center of the stent, or in multiple locations along the length of the stent).

In some embodiments, stent detection (e.g., detecting the spacing between bright points representing probable stent struts in a tomographic image) can be used in place of, as a proxy for, or as a check on, or as a method of computing, the area or diameter of the lumen. In some embodiments, plaque burden (PB) may be tracked instead of or in addition to lumen diameter or area. Plaque burden is the percentage of total vessel area that contains plaque, and is calculated as the difference between the outer wall area of the vessel and the lumen area of the vessel, expressed as a fraction of total vessel area. In some embodiments, the diameter, slope, gradient, or inflection point values that trigger a dog-boning warning are user-editable parameters, although default values may also be provided.

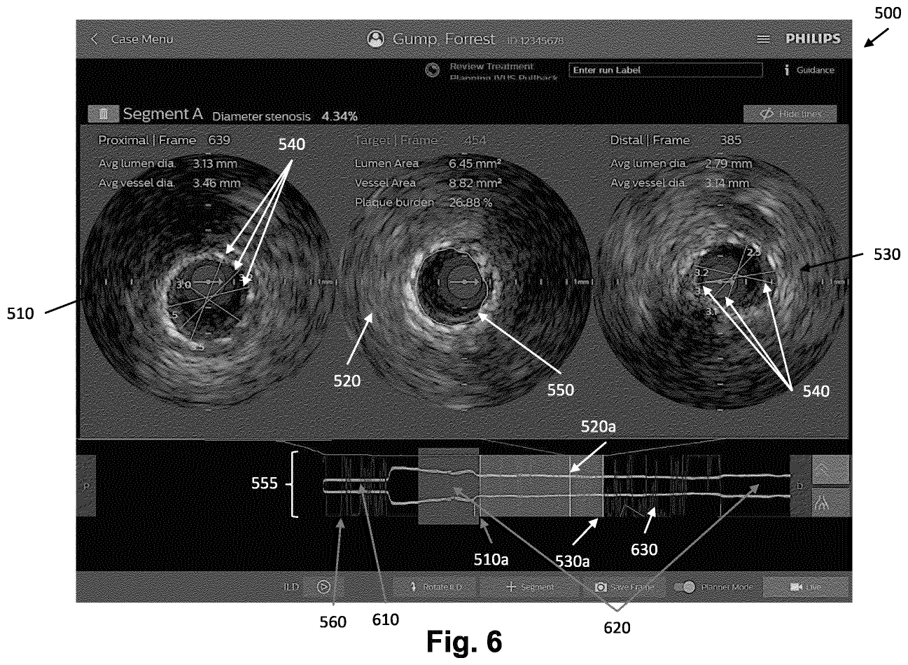

FIG. 6 shows an example screen display 500 of an intraluminal imaging system 100 in accordance with at least one embodiment of the present disclosure. As with FIG. 5, the screen display 500 includes three tomographic images: a proximal reference frame 510, a target frame 520, and a distal reference frame 530. Also visible is a longitudinal display 555 that includes a probability-of-stent graph 560, markers 510a, 520a, and 530a for the positions of the proximal reference frame 510, target frame 520, and distal reference frame 530, and a graphical diameter or area indicator 570.

Stent dilation is accomplished by placing a noncompliant balloon inside the stent 340 and expanding it, section by section, until it is uniformly expanded along its length. Suboptimal coverage occurs when the stents 340 is positioned incorrectly and falls (for example) 2-3 mm short of the margins of a potential lesion.

The longitudinal display 555 shows a region of catheter sheath 610, which can be detected based on a region of high probability-of-stent values 560, coupled with a constant diameter or area that is substantially less than the diameter or area of the proximal reference frame 510 and distal reference frame 530, with a larger lumen diameter occurring outside the edges of the sheath. The sheath is generally not considered in clinical decision making, and so tomographic images that include the sheath may optionally be deleted from the pullback sequence, and their graphical representations may optionally be deleted from the longitudinal display 555.

The longitudinal display 555 also shows two regions of healthy tissue 620 that show evidence of stent under-dilation. This can be detected because the healthy tissue 620 has a low or zero probability-of-stent and a larger diameter or cross-sectional area than the stent region 630, which has a narrower diameter and an intermittently high probability-of-stent. Stent under-dilation may occur for example if the clinician has not yet expanded the stent, or has expanded it suboptimally. Stent under-dilation may be corrected by inserting a noncompliant balloon into the under-dilated section of the stent and inflating it to the desired diameter.

The longitudinal display 555 also shows evidence of anatomical tapering, as the healthy tissue 620 on the left side if the longitudinal display 555 has a larger diameter or cross-sectional area than the healthy tissue 620 on the right side of the longitudinal display. The detection of anatomical tapering is discussed below.

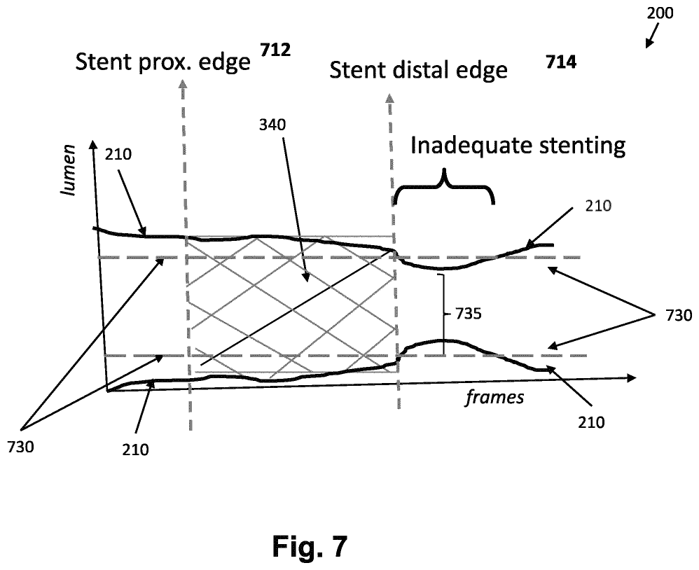

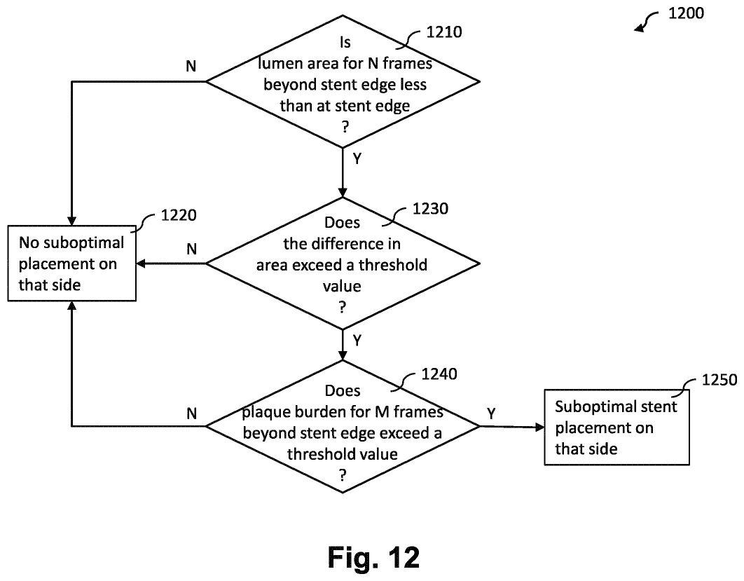

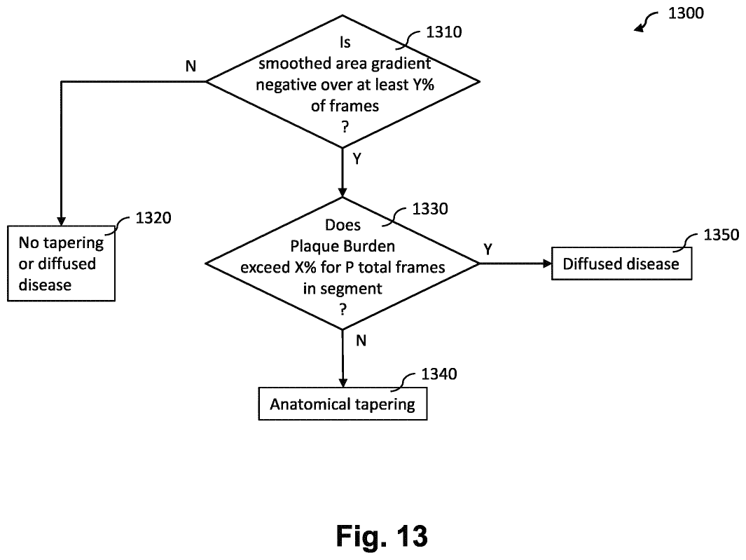

FIG. 7 shows a schematic view of a vessel 200 whose vessel walls 210 have been dilated with a stent 340 having a proximal edge 712 and a distal edge 714, in accordance with aspects of the present disclosure. Also visible is a graphical representation 730 of a plaque burden detection threshold (as applied, for example, in step 1240 of FIG. 12 or step 1330 of FIG. 13, below) that covers the vessel walls 210. In this example, the placement of the stent 340 can be seen to be suboptimal or inadequate, as there is a constriction 735 outside the distal edge 714 of the stent 340, such that the diameter and cross-sectional area of the vessel lumen are smaller outside the edge of the stent 340 than at the edge of the stent, and plaque burden is above a threshold; plaque burden (%)=100×(vessel measurement−lumen measurement)/vessel measurement. Such suboptimal stent placement indicates that the diseased portion (e.g., a stenosis 230 as seen in FIGS. 2 and 3) of the vessel 200 has not been fully covered by the stent 340, either because the stent 340 is too short, or because the stent 340 has not been placed correctly within the vessel 200. Suboptimal stent placement may be corrected for example through placement of an additional stent adjacent to the misplaced stent. The intraluminal treatment anomaly detection system is able to detect this condition on either the proximal side or the distal side of a stent 340, as described below in FIG. 12.

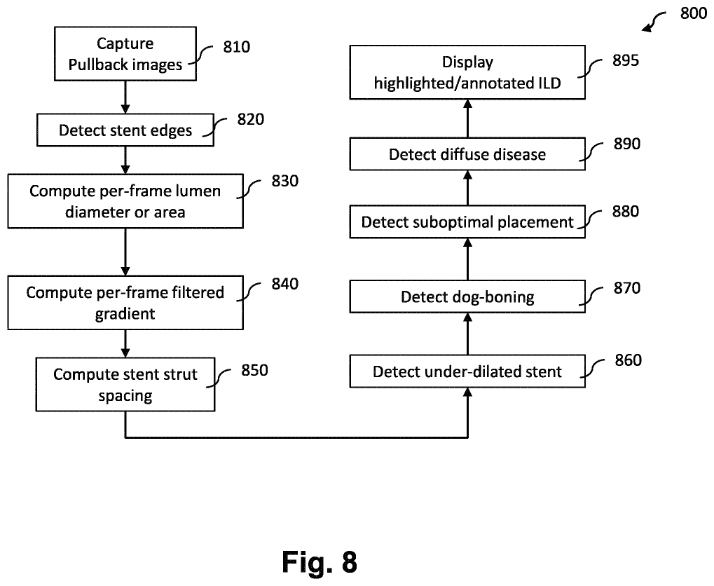

FIG. 8 is a flow diagram illustrating the steps executed by an example intraluminal treatment anomaly detection system 800 in accordance with at least one embodiment of the present disclosure. In step 810, a full set of intraluminal images is captured along the entire length of a pullback.

In step 820, the system detects stent edges, if any, within the imaged portion of the lumen. This may be done for example by using machine learning, image recognition, or pattern recognition to detect probable stent struts within the tomographic image frames, and assigning each frame a probability-of-stent value between 0.0 (definitely no stent) and 1.0 (absolute certainty of stent). In an example, because of image noise and frame-to-frame noise, values of 0.0 and 1.0 may be relatively rare, whereas a region (e.g., a minimum of 10 consecutive frames) where the smoothed probability-of-stent value is consistently under a lower threshold value (e.g., 0.3) may indicate that no stent is present in that region, whereas a region where the smoothed probability-of-stent value is consistently above an upper threshold value (e.g., 0.5) may indicate that a stent is present in that region. The proximal and distal stent edges may then be defined as the locations where the probability-of-stent goes from indicating no stent to indicating the presence of a stent, and the distal stent edges may be defined as the locations where the probability-of-stent goes from indicating a stent to indicating no stent.

In step 830, the system computes and/or otherwise determines per-frame statistics, which may include but are not limited to measurements and/or dimensions associated with the lumen, including lumen diameter, lumen cross-sectional area, stent strut spacing, or plaque burden. In an example, this is done for each frame of the pullback (e.g., for each location in a plurality of locations along the vessel).

In step 840, the system computes at least one filtered gradient-vs.-position curve (e.g., a curve representative of a change in a measurement/dimension along the length of the blood vessel) for at least one per-frame statistic (e.g., lumen diameter). In some embodiments, a graphical representation other than a curve may be used (e.g., a bar graph, stylized drawing, cartoon, or inline longitudinal display) instead of or in addition to a curve. Filtering (e.g., averaging the current frame with the 2, 4, or 6 frames, or other number of frames, before it, after it, or on either side of it) may tend to smooth the gradient curve (or other graphical representation) and prevent image noise or frame-to-frame measurement noise from creating spurious inflection points or gradient values. Other types of filtering may include, but are not limited to, computations based on sampled frames or gated frames. Gating is a means of selecting a frame that corresponds to a particular moment of successive cardiac cycles, or some other way of ensuring the frames represent the region of the frames from which they are selected.

In step 850, some embodiments of the system also compute a filtered curve of detected stent strut spacing.

In step 860, some embodiments of the system detect undilated stents. An undilated or under-dilated stent can be detected based on a region of high probability-of-stent values, coupled with a diameter or area within the stent that is substantially less than the lumen diameter (or area, etc.) outside the edges of the stent.

In step 870, the system detects dog-boning, if present, in any detected stents. Dog-boning can be detected, for example, by detecting the presence of an inflection point in the filtered diameter, area, or stent strut detection across the length of the stent, with slopes on either side of the inflection point exceeding a threshold absolute value. In embodiments where the graphical representation is not a curve, other parameters may be used in place of a slope, such as the difference in value between two bars in a bar graph and/or the sign of difference (positive or negative). Alternatively, or in addition, dog-boning can be detected via analysis of stent strut detection: First, using image recognition, the system identifies the stent structs in each slice. Second, by computing the distance between struts, the system creates a stent strut profile for that slice. Third, the system computes a 3D stent model by comparing the stent strut distance profile across a plurality of frames. The model indicates areas where stent struts are expanded, and where they are not expanded. The 3D visualization can be binary (e.g., based only on whether the distances exceed a threshold value) or continuous, and may for example be visualized through a color map, similar to the gradient display 580 in FIG. 5. By studying the nature of the map, the system can determine if an above-threshold amount of dog-boning is occurring, e.g., is that near the stent edge areas, the stent is much more expanded than the middle of the stent—i.e. the struts are farther apart at the stent edges than in the middle of the stent.

In step 880, the system detects suboptimal coverage, if present, in any detected stents. To do this, the system compares the stent start and end frames (e.g., the proximal and distal edges of the stent) to the profile of the disease in the vicinity of the frames. Specifically, the algorithm compares the stent area to the lumen areas of ±N frames closest to that stent edge. If the lumen area is less than the stent edge area, and the plaque burden (PB) exceeds a threshold amount over a specified number of frames M, then suboptimal coverage has occurred on that side of the stent.

M frames can be quantified by the speed of the pullback in case automatic length measurement is possible, as in the case with co-registration to an angiography image. In an example, M corresponds to a specified distance such as 2-3 mm.

In step 890, the system detects the difference between anatomical tapering and diffuse disease. Anatomical tapering occurs naturally in body lumens such as blood vessels, and can be seen in that more distal frames in the pullback image set will exhibit a gradually decreasing diameter and cross-sectional area as compared with more proximal frames in the pullback. In an anatomically tapering lumen, a filtered curve of diameter or area vs. position within the lumen will not generally exhibit sudden changes in value, but gradually decrease from proximal to distal locations. Conversely, diffuse disease, diffuse disease, or a diffuse lesion occurs when an intermittent or increasing plaque burden is seen within the lumen. For example, a diffuse disease or diffuse lesion can occur with the presence of plaque buildup along a greater length of the vessel as compared to a focal (e.g., localized) lesion. The extent of constriction (e.g., the narrowing of the lumen) can sometimes be relatively less than would be seen in a focal lesion, but the constriction extends for relatively longer along the length of the vessel, and may thus have an equal or greater impact on vessel volume constriction and blood flow constriction. For a tapering lumen, the taper is anatomical (e.g., healthy or normal) if the plaque burden follows a decreasing trend or never increases beyond a specified threshold (e.g., 50%). The taper is indicative of diffuse disease if the plaque burden is above the threshold (e.g., 50%) for a total of P frames (e.g., 20 frames) within a segment, whether continuously or intermittently. If the plaque burden is increasing or intermittent as you move distally, then the tapering is anatomical (e.g., healthy or normal) if the plaque burden never exceeds the threshold amount (e.g., 50%).

In step 895, the system outputs a graphical representation of the detected condition(s) of the vessel to a display. For example, the system creates and displays a longitudinal display 555 (e.g., on the monitor 108) that includes graphs, marks, highlights, color coding, text, and/or numbers sufficient to indicate the suspected presence and locations of various anomalies as described above. This longitudinal display then can be used by a clinician or other user to evaluate the status of a lumen post-treatment, to determine whether treatment is complete or, conversely, whether any additional intervention is called for.

After the system displays the longitudinal display, execution of the method is complete.

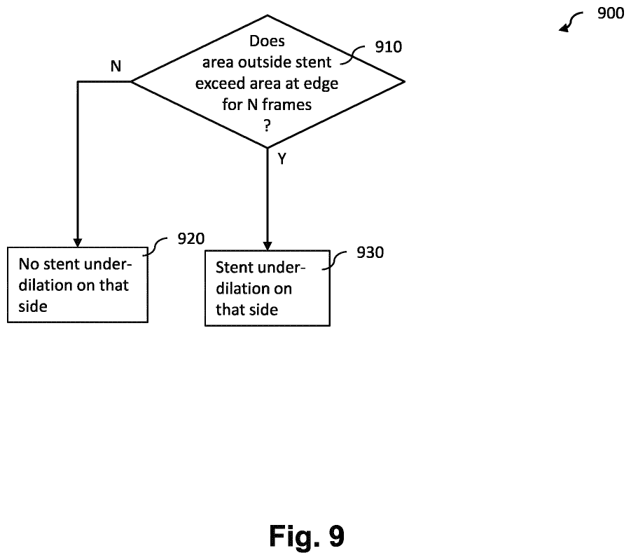

FIG. 9 is a flow diagram 900 of an example stent under-dilation detection algorithm 860 in accordance with at least one embodiment of the present disclosure. In step 910, the algorithm determines whether the lumen area outside the stent exceeds the lumen area of the stent edge, by more than a threshold amount, for a specified number of frames. In a more general sense, other measurements, including but not limited to measured or computed vessel diameter, can be used in place of area for the detection. If yes, execution proceeds to step 920, where the algorithm determines that no stent under-dilation exists on that side of the stent. If no, execution proceeds to step 930, where the algorithm determines that there is stent under-dilation on that side of the stent. In some embodiments, instead of comparing the area inside and outside of the stent, step 910 looks at the filtered gradient of the area for N frames outside of the stent edge, to see if it is expanding. If so, execution proceeds to step 920. If not, execution proceeds to step 930.

In other words, under-dilation is detected when a first value of the lumen dimension (e.g., area or diameter) exceeds a second value of the dimension at the edge of the stent for a specified distance beyond the edge of the stent.

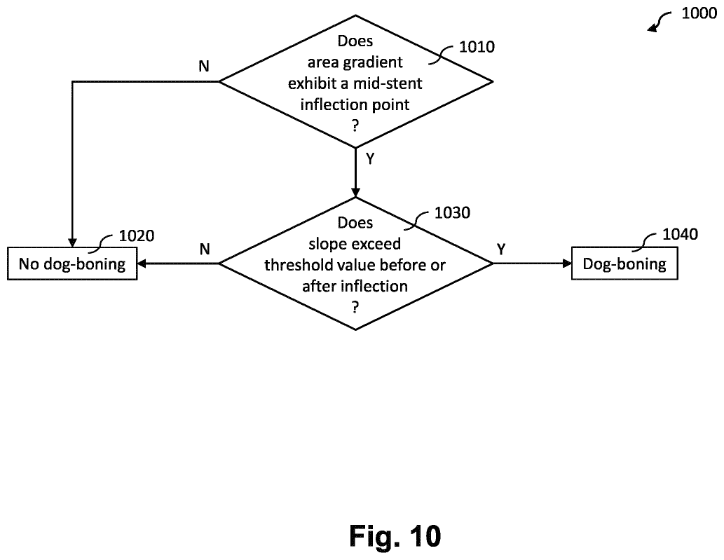

FIG. 10 is a flow diagram 1000 of an example dog-boning detection algorithm 870 in accordance with at least one embodiment of the present disclosure. In step 1010, the algorithm looks at the filtered area gradient in between the proximal and distal edges of the stent, and determines whether an inflection point exists within that range of positions. If no, execution proceeds to step 1020. If yes, execution proceeds to step 1030. In step 1020, the algorithm determines that no dog-boning is present for this particular stent, and execution of the algorithm for that stent is complete. In step 1030 the algorithm determines whether the magnitude or absolute value of the filtered area gradient between the stent proximal and distal edges exceeds a threshold value on either side of the inflection point. If no, execution proceeds to step 1020. If yes, execution proceeds to step 1040, where the algorithm determines that dog-boning is present for this particular stent.

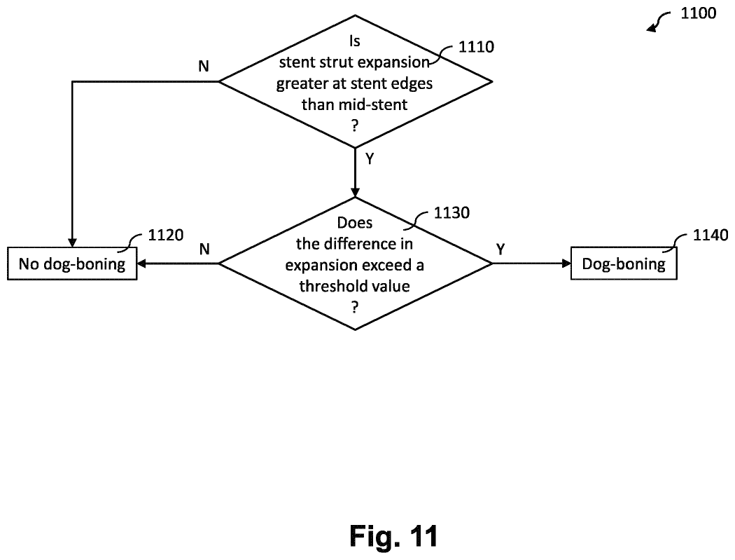

FIG. 11 is a flow diagram 1100 of a different example dog-boning detection algorithm 870 in accordance with at least one embodiment of the present disclosure. In step 1110, the algorithm determines whether stent strut expansion is greater at the edges of the stent than at one or more points within the stent. If no, execution proceeds to step 1120. If yes, execution proceeds to step 1130. In step 1120, the algorithm determines that no dog-boning is present for this particular stent, and execution of the algorithm for that stent is complete. In step 1130, the algorithm determines whether the difference in stent strut expansion exceeds a threshold value. If no, execution proceeds to step 1120. If yes, execution proceeds to step 1140, where the algorithm determines that dog-boning is present for this particular stent.

FIG. 12 is a flow diagram 1200 of an example suboptimal stent placement detection algorithm 880 in accordance with at least one embodiment of the present disclosure. In step 1210, the algorithm determines whether the lumen area for at least N frames outside the stent edge is less than the lumen area of the stent edge. If no, execution proceeds to step 1220. If yes, execution proceeds to step 1230. In step 1220, the algorithm determines that no suboptimal placement is detected for this particular side of this particular stent, and execution of the algorithm for that side of that stent is complete. In step 1230, the algorithm determines whether the difference in area outside the stent vs. the stent edge exceeds a threshold value (e.g., 0.3 mm²). If no, execution proceeds to step 1220. If yes, execution proceeds to step 1240. In step 1240, the algorithm determines whether the plaque burden outside the stent edge exceeds a threshold value (e.g., 50%) for at least M frames (e.g., 20 frames). If no, execution proceeds to step 1220. If yes, execution proceeds to step 1250, where the algorithm determines that there is suboptimal placement for this stent on this side.

In other words, suboptimal stent placement or incomplete coverage of a lesion is detected when for a first distance beyond an edge of the stent, a first value of the dimension is less than a second value of the dimension at the edge of the stent by at least a threshold amount, and the plaque burden for a second distance beyond the edge of the stent exceeds a threshold value.

FIG. 13 is a flow diagram 1300 of an example anatomical tapering vs. diffuse disease detection algorithm 890 in accordance with at least one embodiment of the present disclosure. In step 1310, the algorithm determines, for a length of non-stented tissue, whether the smoothed area gradient is negative across a threshold percentage of frames (e.g., 51%). If no, execution proceeds to step 1320. If yes, then the algorithm has detected either diffuse disease or anatomical tapering to be present in the vessel, and execution proceeds to step 1330. In step 1320, the algorithm determines that no anatomical tapering or diffuse disease is detected for this particular lumen segment, and execution of the algorithm for that lumen segment is complete. In step 1330, the algorithm determines whether the plaque burden exceeds a threshold value (e.g., 50%) within a threshold number or percentage of frames within the segment. If no, execution proceeds to step 1340, where the algorithm determines that the vessel is narrowing due to anatomical tapering. If yes, execution proceeds to step 1350, where the algorithm determines that the vessel is narrowing due to diffuse disease.

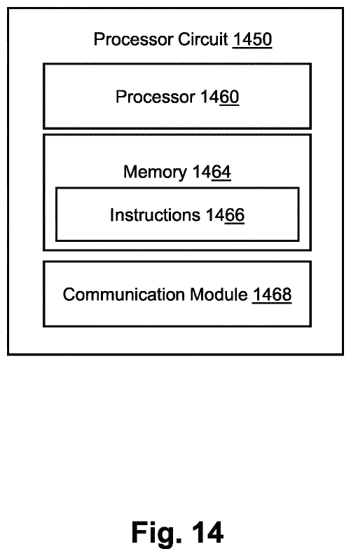

FIG. 14 is a schematic diagram of a processor circuit 1450, according to embodiments of the present disclosure. The processor circuit 1450 may be implemented in the ultrasound imaging system 100, or other devices or workstations (e.g., third-party workstations, network routers, etc.) as necessary to implement the method. As shown, the processor circuit 1450 may include a processor 1460, a memory 1464, and a communication module 1468. These elements may be in direct or indirect communication with each other, for example via one or more buses.

The processor 1460 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 860 may also comprise another hardware device, a firmware device, or any combination thereof configured to perform the operations described herein. The processor 1460 may also be implemented as a combination of computing devices, e.g., a combination of a DSP and a microprocessor, a plurality of microprocessors, one or more microprocessors in conjunction with a DSP core, or any other such configuration.

The memory 1464 may include a cache memory (e.g., a cache memory of the processor 1460), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 1464 includes a non-transitory computer-readable medium. The memory 1464 may store instructions 1466. The instructions 1466 may include instructions that, when executed by the processor 1460, cause the processor 1460 to perform the operations described herein. Instructions 866 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 1468 can include any electronic circuitry and/or logic circuitry to facilitate direct or indirect communication of data between the processor circuit 1450, and other processors or devices. In that regard, the communication module 1468 can be an input/output (I/O) device. In some instances, the communication module 868 facilitates direct or indirect communication between various elements of the processor circuit 1450 and/or the ultrasound imaging system 100. The communication module 1468 may communicate within the processor circuit 1450 through numerous methods or protocols. Serial communication protocols may include but are not limited to US SPI, I²C, RS-232, RS-485, CAN, Ethernet, ARINC 429, MODBUS, MIL-STD-1553, or any other suitable method or protocol. Parallel protocols include but are not limited to ISA, ATA, SCSI, PCI, IEEE-488, IEEE-1284, and other suitable protocols. Where appropriate, serial and parallel communications may be bridged by a UART, USART, or other appropriate subsystem.

External communication (including but not limited to software updates, firmware updates, preset sharing between the processor and central server, or readings from the ultrasound device) may be accomplished using any suitable wireless or wired communication technology, such as a cable interface such as a USB, micro USB, Lightning, or FireWire interface, Bluetooth, Wi-Fi, ZigBee, Li-Fi, or cellular data connections such as 2G/GSM, 3G/UMTS, 4G/LTE/WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

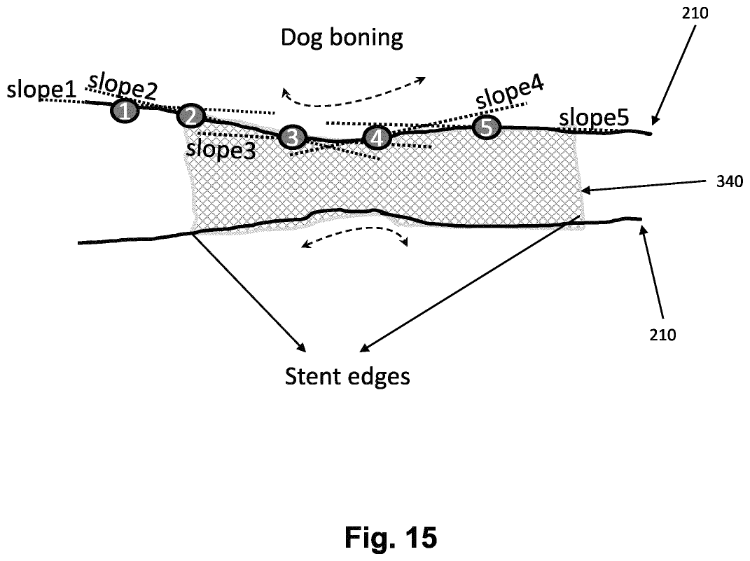

FIG. 15 shows a schematic view of a vessel whose vessel walls 210 have been dilated with a stent 340 that exhibits dog-boning, in accordance with aspects of the present disclosure. By comparing the slopes of the stent at various points along the profile (e.g., slope 1 at point 1, slope 2 at point 2, slope 3 at point 3, slope 4 at point 4, and slope 5 at point 5), the algorithm can identify an inflection point within the stent and thus detect dog-boning as described above in FIG. 10. In this example, slopes 1 and 5 are approximately equal, whereas slopes 2 and 4 have opposite sign (negative and positive slope), and point 3, with an absolute slope value smaller than slopes 2 and 4, is the inflection point. This pattern shows dog-boning.

A number of variations are possible on the examples and embodiments described above. For example, the intraluminal treatment anomaly detection system may be employed in anatomical systems within the body other than those described, or may be employed to image other disease types, object types, or procedure types than those described. The technology described herein may be applied to intraluminal imaging sensors of diverse types, whether currently in existence or hereinafter developed. The analysis described above can also be performed using, instead of areas, volumes, measured or computed mean diameters or intrinsic diameters, or any other variable representative of vessel dimensions at different points along the vessel. The analysis can be performed with an evenly or regularly sampled subset of measurements rather than using all measurements all measurements, as long as the measurements don't reflect high local variance, in which case a smoothening filter may be included. Co-registration with a different modality such as angiography can be used to indicate location or severity of these above-identified anomalies on the angiogram image itself. Any of the thresholds, ranges, or numbers of frames described above may be user-editable quantities, although defaults may also be supplied by the system. In order to speed up execution or reduce computing burden, the system may function with only a sampling of frames (e.g., every fifth frame) rather than with the entire image dataset.

Accordingly, the logical operations making up the embodiments of the technology described herein are referred to variously as operations, steps, objects, elements, components, or modules. Furthermore, it should be understood that these may occur or be performed in any order, unless explicitly claimed otherwise or a specific order is inherently necessitated by the claim language. Steps may be added, deleted, combined, or rearranged without departing from the spirit of the present disclosure. All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the intraluminal treatment anomaly detection system. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” Unless otherwise noted in the claims, stated values shall be interpreted as illustrative only and shall not be taken to be limiting.

The above specification, examples and data provide a complete description of the structure and use of exemplary embodiments of the intraluminal treatment anomaly detection system as defined in the claims. Although various embodiments of the claimed subject matter have been described above with a certain degree of particularity, or with reference to one or more individual embodiments, those skilled in the art could make numerous alterations to the disclosed embodiments without departing from the spirit or scope of the claimed subject matter. Still other embodiments are contemplated. It is intended that all matter contained in the above description and shown in the accompanying drawings shall be interpreted as illustrative only of particular embodiments and not limiting. Changes in detail or structure may be made without departing from the basic elements of the subject matter as defined in the following claims.

What is claimed is:

1. An intravascular imaging system, comprising:
a processor circuit configured for communication with an intravascular imaging catheter sized and shaped for positioning within a lumen of a blood vessel, wherein the processor circuit configured to:
receive a plurality of intravascular images obtained by the intravascular imaging catheter while the intravascular imaging catheter is positioned within the lumen, wherein the plurality of intravascular images corresponds to a plurality of locations along a length of the blood vessel;
determine a measurement associated with the lumen for each image of the plurality of intravascular images;
generate a first graphical representation representative of a change in the measurement along the length of the blood vessel;
detect a condition of the blood vessel based on the first graphical representation; and
output, to a display in communication with the processor circuit, a second graphical representation representative of the condition.

2. The system of claim 1, wherein the processor circuit determining the measurement comprises:
averaging, for a location of the plurality of locations, a quantity of the measurement at the location and the quantity of the measurement at another location of the plurality of locations.

3. The system of claim 1, wherein the processor circuit determining the measurement comprises the processor circuit computing at least one of a cross-sectional area of the lumen or a diameter of the lumen.

4. The system of claim 1, wherein the processor circuit detecting the condition comprises the processor circuit detecting at least one of an anatomical tapering of the blood vessel or a diffuse disease of the blood vessel.

5. The system of claim 4, wherein the condition comprises the anatomical tapering, and wherein the processor circuit detecting the condition comprises the processor circuit detecting that a plaque burden of the blood vessel does not exceed a threshold value for a number of locations within a segment of the blood vessel.

6. The system of claim 4, wherein the condition comprises the diffuse disease, and wherein the processor circuit detecting the condition comprises the processor circuit detecting that a plaque burden of the vessel exceeds a threshold value for a number of locations within a segment of the blood vessel.

7. The system of claim 1, wherein one or more of the plurality of intravascular images comprises a stent positioned within the lumen, and wherein the processor circuit detecting the condition of the blood vessel comprises detecting a post-treatment condition.

8. The system of claim 7, wherein the measurement comprises a spacing between struts of the stent.

9. The system of claim 7, wherein the processor circuit detecting the condition comprises the processor circuit detecting at least one of dog-boning of the stent, under-dilation of the stent, or incomplete coverage of a lesion by the stent.

10. The system of claim 9, wherein the condition is the dog-boning of the stent, and wherein the processor circuit detecting the condition comprises the processor circuit determining that a rate of change of the measurement exhibits an inflection point within the stent, and that the rate of change of the measurement within the stent exceeds a threshold value proximal to or distal to the inflection point.

11. The system of claim 9, wherein the condition is the under-dilation of the stent, and wherein the processor circuit detecting the condition comprises processor circuit determining that a first value of the measurement exceeds a second value of the measurement at an edge of the stent by more than a threshold amount for a distance beyond the edge of the stent.

12. The system of claim 9, wherein the condition is the incomplete coverage of the lesion by the stent, and wherein the processor circuit detecting the condition comprises detecting that:
for a first distance beyond an edge of the stent, a first value of the measurement is less than a second value of the measurement at the edge of the stent by at least a threshold amount; and
a plaque burden for a second distance beyond the edge of the stent exceeds a threshold value.

13. The system of claim 1,
wherein the processor circuit is configured to receive an extravascular image of the blood vessel and to co-register the plurality of intravascular images to the plurality of locations along the length of the vessel in the extravascular image, and
wherein the processor circuit outputting the second graphical representation representative of the condition comprises the processor circuit outputting an indication of the condition along the length of the vessel in the extravascular image.

14. The system of claim 1, further comprising:
the intravascular imaging catheter, wherein the intravascular imaging catheter comprises an intravascular ultrasound (IVUS) imaging catheter.

15. An intravascular imaging method, comprising:
receiving, at a processor circuit in communication with an intravascular imaging catheter, a plurality of intravascular images obtained by the intravascular imaging catheter while the intravascular imaging catheter is positioned with a lumen a blood vessel, wherein the plurality of intravascular images corresponds to a plurality of locations along a length of the blood vessel;
determining, by the processor circuit, a measurement associated with the lumen for each image of the plurality of intravascular images;
generating, by the processor circuit, a first graphical representation representative of a change in the measurement along the length of the blood vessel;
detecting, by the processor circuit, a condition of the blood vessel based on the first graphical representation; and
outputting, to a display in communication with the processor circuit, a second graphical representation representative of the condition.

16. An intravascular ultrasound (IVUS) imaging system, comprising:
an IVUS imaging catheter sized and shaped for positioning within a lumen of a blood vessel; and
a processor circuit configured for communication with the IVUS imaging catheter, wherein the processor circuit configured to:
receive a plurality of IVUS images obtained by the IVUS imaging catheter while the IVUS imaging catheter is positioned within the lumen, wherein the plurality of IVUS images corresponds to a plurality of locations along a length of the blood vessel;
determine a measurement associated with the lumen for each image of the plurality of IVUS images;
generate a curve representative of a change in the measurement along the length of the blood vessel;
detect a condition of the blood vessel based on the curve, wherein the condition comprises at least one of dog-boning of a stent within the blood vessel, under-dilation of the stent, incomplete coverage of a lesion of the blood vessel by the stent, diffuse disease of the blood vessel, or anatomical tapering of the blood vessel; and
output, to a display in communication with the processor circuit, a graphical representation representative of the condition.
